(19) United States
(12) Patent Application Publication
(10) Pub. No.: US 2023/0103788 A1
(43) Pub. Date: Apr. 6, 2023

(54) APPARATUS AND METHOD FOR SENSORY SUBSTITUTION AND MULTI-PATH TRANSMISSION OF VISUAL INFORMATION

(71) Applicant: ELECTRONICS AND TELECOMMUNICATIONS RESEARCH INSTITUTE, Daejeon (KR)

(72) Inventors: Moo Seop KIM, Daejeon (KR); Chi Yoon JEONG, Daejeon (KR); Kyeong Deok MOON, Daejeon (KR); Yun Kyung PARK, Daejeon (KR)

(21) Appl. No.: 17/952,493

(22) Filed: Sep. 26, 2022

(30) Foreign Application Priority Data
Oct. 1, 2021 (KR) 10-2021-0130969
Jul. 4, 2022 (KR) 10-2022-0081959

(51) Int. Cl. G06F 3/01

(52) U.S. Cl. CPC G06F 3/01; A61F 9/08

(57) ABSTRACT

The present invention relates to an apparatus and method for sensory substitution and multi-path transmission of visual information. The apparatus for sensory substitution and multi-path transmission of visual information according to the present invention includes a target information extraction module configured to extract information of which a type corresponds to a type requested by a user from image information received from an external device on the basis of user input information, a sensory pathway determination module configured to determine one or more sensory pathways through which the information extracted based on the user input information is transmitted, a substitution signal generation module configured to convert the extracted information to generate substitution signals respectively corresponding to the one or more sensory pathways, and a control and output module configured to output the substitution signals through a sensory signal transmission device of the user.

### CROSS-REFERENCE TO RELATED APPLICATIONS

This application claims priority to and the benefit of Korean Patent Applications No. 10-2021-0130969, filed on Oct. 1, 2021, and No. 10-2022-0081959, filed on Jul. 4, 2022, the disclosures of which are incorporated herein by reference in its entirety.

### BACKGROUND

### 1. Field of the Invention

The present invention relates to an apparatus and method for sensory substitution and transmission which are capable of enabling a user's brain to perceive information on a specific sense from a converted signal by substituting and transmitting the information on the specific sense with information that can be sensed by another sensory organ on the basis of brain plasticity. The present invention includes not only a technique for simply substituting sensory information, but also a technique and method for transmitting visual information with various sensory signals so that the user can have an augmented sense.

### 2. Description of Related Art

Generally, sensory substitution is a method converting and transmitting sensory information of a body whose function is damaged or deteriorated due to an accident or aging into other sensory signals so that a user who has undergone a training process for a certain period can perceive the original sensory information through a substituted signal. It is known that such sensory substitution technology is based on the characteristics of a human brain that is structurally or functionally changed and reorganized, that is, brain plasticity, to adapt to a new environment.

It is known that about 80% of information that a person perceives from the outside in daily life is achieved through a visual sense. Therefore, the visual sense is a sense that is felt through the most important information collecting organ among sensory organs of the human body, and when the visual sense is lost, problems such as inconvenience in walking, difficulty in understanding the situation, and the like occur. In order to cope with such problems, various studies for converting and transmitting visual information into other sensory signals have been conducted.

The conventional methods for converting visual information into other sensory signals include auditory substitution technology for converting visual information into auditory signals, tactile substitution technology for converting visual information into tactile signals, and the like. The conventional auditory substitution technology or tactile substitution technology is mainly technology for converting an input image into a black-and-white image or a gray image through pre-processing and then converting and transmitting the converted image into other sensory signals rather than visual signals according to predefined rules.

vOICe, which is the most representative technology in auditory substitution technology, is technology for converting an input image into a two-dimensional image with a 64×64 resolution in grayscale, then dividing a frame of the image into 64 columns with respect to a time axis, mapping a position and brightness information of pixels in each column to a frequency, an amplitude, etc. of sound, generating sound information, and then transmitting the generated sound information. In addition, EyeMusic in auditory substitution technology is technology for, in order to solve a problem of loss of color information, which is an important element in the image, converting an image or a video into an image in grayscale and processing the converted image, grouping the same color areas in the input image, mapping each color to a different instrument sound, and transmitting the mapped sound with sound signals.

Generally, visual information perceived by humans includes various types of information such as a shape including an outline, a position of an object, a distance, color, texture, a path, a direction, presence or absence of light, and mood or emotion. However, in the conventional auditory substitution or tactile substitution technology, since information limited to a shape, color, or the like, among pieces of visual information, is provided by being substituted with other sensory information, various pieces of visual information cannot be transmitted. Specifically, in the conventional sensory substitution technology for substituting and transmitting visual information with other sensory signals, since a video or an image is converted in a predetermined way and transmitted to a single sensory organ, there is a problem in that various pieces of visual information cannot be effectively transmitted to the user.

Further, since the conventional sensory substitution technology follows a simple signal transmission method, there is a problem in that the visual information required by the user cannot be selectively transmitted.

Meanwhile, recently, in a virtual/augmented environment represented as non-face-to-face society and the metaverse that has emerged due to Coronavirus disease 2019 (COVID-19), there is an increasing need for technologies for efficiently transmitting visual information and for augmenting or improving the user's senses. However, the above-described sensory substitution technologies have a limitation in that only an indirect path for simply providing the lost functionality can be provided.

Further, in the case of most sensory information, the amount of information that sensory receptors can sense may be limited depending on the characteristics of sensory organs. In the conventional sensory substitution technology, since a single piece of substitution sensory information is used (transmitted through a single path), there is a limit in faithfully transmitting visual information of a video or image.

### SUMMARY OF THE INVENTION

The present invention is directed to solving the above problems and limitations, and providing an apparatus and method for sensory substitution and multi-path transmission of visual information, which are capable of converting a plurality of pieces of visual information that a user wants to receive from, among various pieces of visual information included in a video or image, into other sensory signals suitable for the characteristics of the plurality of pieces of visual information and transmitting the converted sensory signals to a plurality of sensory organs to efficiently transmit the visual information to the user.

In particular, the main purpose of the conventional auditory or tactile substitution technologies was to help the visually impaired walk or live a daily life, whereas the present invention is directed to providing an apparatus and method for sensory substitution and multi-path transmission of visual information, which are capable of efficiently transmitting visual information in military operations that require the transmission of confidential information and in extreme working environments in the basement or late at night, as well as to help the existing visually impaired, or augmenting and transmitting visual information in order to maximize the user's experience in virtual reality or augmented reality represented by the metaverse.

Objects of the present invention are not limited to the above-described objects and other objects that are not described will be clearly understood by those skilled in the art from the following descriptions.

According to an aspect of the present invention, there is provided an apparatus for sensory substitution and multi-path transmission of visual information, including a target information extraction module configured to extract information of which a type corresponds to a type requested by a user from image information received from an external device on the basis of user input information, a sensory pathway determination module configured to determine one or more sensory pathways through which the information extracted based on the user input information is transmitted, a substitution signal generation module configured to convert the extracted information to generate substitution signals respectively corresponding to the one or more sensory pathways, and a control and output module configured to output the substitution signals through a sensory signal transmission device of the user. The type is included in the user input information.

The one or more sensory pathways may include a plurality of sensory pathways, and the substitution signal generation module may convert the extracted information to generate substitution signals respectively corresponding to the plurality of sensory pathways.

The plurality of sensory pathway may include sensory pathways corresponding to at least two senses of a visual sense, an auditory sense, a tactile sense, and a heat sensation.

The target information extraction module may analyze the image information to generate overall visual information including at least one of shape information, spatial information, global information, and complex information, and extract information of which a type corresponds to the type from the overall visual information on the basis of the user input information.

The control and output module may synchronize the substitution signals respectively corresponding to the plurality of sensory pathways on the basis of a frame of the image information, and output the synchronized substitution signals through the sensory signal transmission device.

The control and output module may recognize the user's action on the basis of the image information, correct characteristics of the substitution signals on the basis of the user's action, and output the corrected substitution signals through the sensory signal transmission device.

The control and output module may correct characteristics of the substitution signals on the basis of a position of an input device of the image information and a position of the sensory signal transmission device, and output the corrected substitution signals through the sensory signal transmission device.

The user input information may include information on a transmission strength. In this case, the control and output module may correct a strength of the substitution signal according to the transmission strength, and output the corrected substitution signals through the sensory signal transmission device.

The control and output module may calculate a time required for the substitution signal generation module to convert the extracted information into the substitution signals. In this case, the substitution signal generation module may determine whether the conversion of the extracted information into the substitution signals is to be simplified on the basis of the time.

According to another aspect of the present invention, there is provided a method for sensory substitution and multi-path transmission of visual information, including extracting information of which a type corresponds to a type requested by a user from image information received from an external device on the basis of user input information, determining one or more sensory pathways through which the information extracted based on the user input information is transmitted, converting the extracted information and generating substitution signals respectively corresponding to the one or more sensory pathways, and outputting the substitution signals through a sensory signal transmission device of the user.

The one or more sensory pathways may include a plurality of sensory pathways. In this case, in the generating of the substitution signals, the extracted information may be converted and substitution signals respectively corresponding to the plurality of sensory pathways may be generated.

The plurality of sensory pathways may include sensory pathways corresponding to at least two senses of a visual sense, an auditory sense, a tactile sense, and a heat sensation.

The method for sensory substitution and multi-path transmission of visual information may further include, after the generating of the substitution signal, a substitution signal synchronization operation of synchronizing the substitution signals on the basis of a frame of the image information.

The method for sensory substitution and multi-path transmission of visual information may further include, after the generating of the substitution signal, a substitution signal correction operation of correcting characteristics of the substitution signals on the basis of a position of an input device of the image information and a position of the sensory signal transmission device. In this case, in the outputting of the substitution signal, the corrected substitution signals may be output through the sensory signal transmission device.

In the extracting of the information of which the type corresponds to the type requested by the user, the image information may be analyzed and overall visual information including at least one of shape information, spatial information, global information, and complex information may be generated, and the information of which the type corresponds to the type may be extracted from the overall visual information on the basis of the user input information.

In the outputting of the substitution signal, the substitution signals respectively corresponding to the plurality of sensory pathways may be synchronized on the basis of a frame of the image information, and the synchronized substitution signals may be output through the sensory signal transmission device.

The user input information may include information on a transmission strength. In this case, in the generating of the substitution signal, a strength of the substitution signal may be corrected by reflecting the transmission strength.

In the generating of the substitution signal, a time required to convert the extracted information into the substitution signals may be calculated, and whether the conversion of the extracted information into the substitution signals is to be simplified may be determined based on the time, the extracted information is converted based on a result of the determination, and the substitution signals may be generated.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a block diagram illustrating a configuration of an apparatus for sensory substitution and multi-path transmission of visual information according to an embodiment of the present invention;

FIG. 2 is a block diagram illustrating a detailed configuration of the apparatus for sensory substitution and multi-path transmission of visual information according to the embodiment of the present invention;

FIG. 3 is an exemplary diagram illustrating a concept of sensory substitution and multi-path transmission of visual information according to the present invention;

FIG. 4 is a block diagram illustrating a configuration of an apparatus for sensory substitution and multi-path transmission of visual information according to another embodiment of the present invention;

FIGS. 5A and 5B illustrate flowcharts for describing a method for sensory substitution and multi-path transmission of visual information according to an embodiment of the present invention; and

FIG. 6 is a block diagram illustrating a computer system for implementing the method according to the embodiment of the present invention.

### DETAILED DESCRIPTION OF EXEMPLARY EMBODIMENTS

Visual information perceived by human eyes is divided into shape information including shapes and color, presence or absence of light, and texture of objects present in an image, spatial information in a geometrical aspect, such as positions, distances, and the directions, global information such as color and shapes extracted from an entire image area, including objects and backgrounds present in the image, and complex information including path, emotion, mood, and the like obtained by using a plurality of pieces of information such as shape information, spatial information, global information, and the like. However, since the conventional sensory substitution devices that convert visual information into information on other senses mainly convert and transmit two-dimensional color image information, there are disadvantages in that it is difficult to transmit a sense of distance to the user, or it is difficult for the user to efficiently receive various pieces of visual information by converting the entire input image in a predetermined way and transmitting the converted image to a single sensory organ. Further, since the conventional sensory substitution technology was mainly developed for the purpose of transmitting information for the visually impaired, it is difficult to apply the conventional sensory substitution technology to various fields.

The sensory substitution technology for various pieces of visual information included in a video or an image may be used by the user to efficiently perceive a surrounding environment not only for helping the visually impaired, but also in military fields such as infiltrating enemy camps or reconnaissance, in extreme environments such as in the absence of lighting due to power outages due to underground or accidents, and late-night work, or in extreme environments in which dangerous situations analyzed with visual information in general industrial sites are safely transmitted to workers. Further, the sensory substitution technology may be used to synchronize the converted information according to the transmitted visual information and transmit the synchronized information through various paths so that the user can feel the visual information more realistically in virtual reality or augmented reality.

According to the present invention, after receiving a plurality of pieces of visual information that the user wants to receive from and extracting information requested by the user from various pieces of visual information such as shape information, spatial information, global information, and complex information that are included in the collected image, the information may be efficiently transmitted to the user by converting the extracted information into other sensory signals suitable for the characteristics of the extracted information and transmitting the information to a plurality of sensory organs.

An apparatus and method for transmitting various pieces of visual information through multiple paths according to the present invention will be described with reference to embodiments in which visual information is converted into an auditory signal, a tactile signal, and a heat sensation signal. However, the apparatus and method of the present invention are not limited to the conversion method according to the embodiments and may be applied to other sensory information.

Advantages and features of the present invention and methods of achieving the same will be clearly understood with reference to the accompanying drawings and embodiments described in detail below. However, the present invention is not limited to the embodiments to be disclosed below but may be implemented in various different forms. The embodiments are provided in order to fully explain the present embodiments and fully explain the scope of the present invention for those skilled in the art. The scope of the present invention is only defined by the appended claims. Meanwhile, terms used in this specification, are considered in a descriptive sense only and not for purposes of limitation. In this specification, the singular forms include the plural forms unless the context clearly indicates otherwise. It will be understood that the terms “comprise” and/or “comprising” when used herein, specify some stated components, steps, operations and/or elements, but do not preclude the presence or addition of one or more other components, steps, operations and/or elements.

In this specification, the term “request information” is referred to as information on a type of an object that the user is interested in or information indicating a type of visual information that a user wants to receive from image information.

In this specification, the term “request sensory information” is information that a user requests for a sense intended to receive the request information, and is referred to as information in which the user's intention is expressed with respect to a sense that is a medium for transmitting the request information.

In this specification, the term “transmission strength information” is referred to as information expressing a transmission strength of a sense.

In this specification, the term “visual information” is used to collectively refer to information generated based on image information, and may include shape information, spatial information, global information, and complex information. The visual information referred to in this specification includes information that can be recognized through a visual sense, such as color or outline, as well as information that can be obtained through analysis of image information, such as a distance between a user and an object.

In this specification, the term “target information” is referred to as visual information that meets request information of the user.

In this specification, the term “sensory signal” is referred to as a signal transmitted by the apparatus for sensory substitution and multi-path transmission according to the present invention to a sensory organ of the user.

In this specification, the term “substitution signal” is referred to as a sensory signal that substitutes for the target information. The substitution signal is generated by the apparatus for sensory substitution and multi-path transmission of visual information according to the present invention in order to convert the target information into another form of information and transmit the converted information to the sensory organs of the user. For example, when the target information is visual information such as “color of a ball,” the substitution signal thereof may be a heat sensation signal indicating a predetermined temperature determined according to “the color of the ball.”

In this specification, the term “sensory organ” is an organ of a human body that senses and receives external stimuli, and is referred to as an organ composed of various receptors that receive senses such as a tactile sense, a temperature sensation, a cold sensation, a sense of pain, a proprioceptive sense, and the like.

In this specification, the term “sensory pathway” is referred to as a path through which nerve impulses follow from the sensory organ of the user to the sensory area of the user's brain. In this specification, the term “determining a sensory pathway” is referred to as determining a sensory organ of a user to which target information or a substitution signal is to be received.

In descriptions of the present invention, when detailed descriptions of related known configurations or functions are deemed to unnecessarily obscure the gist of the present invention, they will be omitted

Before describing the embodiments of the present invention, a core operation of the present invention will be described. The core operation of the present invention consists of four operations S1 to S4.

Operation S1 is an operation of generating target information on the basis of user input information and image information. Specifically, the apparatus for sensory substitution and multi-path transmission of visual information according to the present invention generates visual information (hereinafter, referred to as the “target information”) that meets the user's request from the image information received from an external device, on the basis of the user input information. The target information may be one piece of information or a plurality of pieces of information. That is, the apparatus for sensory substitution and multi-path transmission of visual information according to the present invention generates at least one piece of target information on the basis of the user input information and the image information. More specifically, the apparatus for sensory substitution and multi-path transmission of visual information according to the present invention extracts request information from the user input information, and determines a type of visual information desired by the user on the basis of the request information. The apparatus extracts visual information that meets the determined type from the image information to generate the target information.

Operation S2 is an operation of determining at least one sensory pathway through which the target information is transmitted, on the basis of the user input information. Specifically, the apparatus for sensory substitution and multi-path transmission of visual information according to the present invention parses/extracts request sensory information from the user input information to determine a sensory pathway through which the target information is transmitted. The apparatus determines one or more sensory pathways through which the target information is transmitted.

Operation S3 is an operation of converting the target information into a substitution signal corresponding to the sensory pathway determined in operation S2.

Operation S4 is an operation of outputting the substitution signal through a sensory signal transmission device of the user.

Hereinafter, embodiments of the present invention will be described in detail with reference to the accompanying drawings. In describing the present invention, like reference numerals may refer to like parts or components regardless of the reference numerals for the ease of the overall understanding.

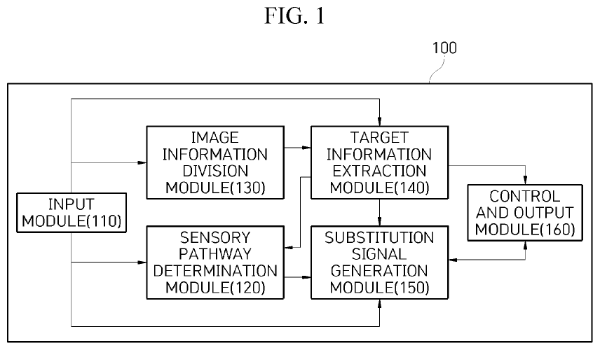

FIG. 1 is a block diagram illustrating a configuration of an apparatus 100 for sensory substitution and multi-path transmission of visual information according to an embodiment of the present invention.

The apparatus 100 for sensory substitution and multi-path transmission of visual information according to the embodiment of the present invention includes an input module 110, a sensory pathway determination module 120, an image information division module 130, a target information extraction module 140, a substitution signal generation module 150, and a control and output module 160. The apparatus 100 for sensory substitution and multi-path transmission of visual information illustrated in FIG. 1 is an apparatus according to an embodiment, and the components of the apparatus 100 are not limited to the embodiment illustrated in FIG. 1, and as necessary, other components may be added or the components of the apparatus 100 may be integrated, changed, or removed.

The input module 110 receives user input information and image information from an external device. The user input information may include request information. The request information may include information on a type of an object that the user is interested in or information indicating a type of visual information that the user wants to receive from the image information. Examples of request information regarding the type of the object may include a person, a car, an animal, etc., and examples of request information regarding the type of the visual information may include a shape, a position, a distance, a moving direction, a color, etc. Examples of request information in which the type of the object and the type of the visual information are mixed may include a moving direction of an animal, a location of a vehicle, a distance from a person, etc.

The user input information may further include at least one of information (hereinafter, referred to as “request sensory information”) on a sense to receive the request information and information (hereinafter referred to as “transmission strength information”) expressing a transmission strength of the sense. Examples of the request sensory information may include a visual sense, an auditory sense, a pressure, a vibration, a heat sensation, etc. Examples of the transmission strength information may include a high level, a medium level, a low level, etc.

The user input information may include at least one of the request information, the request sensory information, and the transmission strength information, or a combination thereof. The request sensory information and the request information may be used by the sensory pathway determination module 120 to determine the sensory pathway through which the visual information (target information) that meets the user's request is transmitted to the user. In other words, the request sensory information and the request information may be used by the sensory pathway determination module 120 to determine which sensory signal is used to transmit the target information corresponding to the request information to the user. The request sensory information and the transmission strength information that are included in the user input information are information for personalization for the user. Sensory signals that are easy to perceive may be different depending on users, this is because a sensitivity may also be different for each sensory signal.

The input module 110 may receive the user input information in various ways. For example, the input module 110 may receive the user input information through a button or text input. The input module 110 may separately extract the request information, the request sensory information, and the transmission strength information from the user input information. For example, the input module 110 may parse the user input information to obtain the request information, the request sensory information, or the transmission strength information.

The image information received by the input module 110 may include at least one of a still image and a continuous image (video). Further, the image information may be an image (i.e., color image, depth image, or thermal image) obtained through a red-green-blue (RGB) camera, a three-dimensional (3D) depth camera, a thermal imaging camera, or the like. The input module 110 transmits the image information to the image information division module 130. Further, the input module 110 transmits the user input information to the sensory pathway determination module 120, the target information extraction module 140, and the substitution signal generation module 150. The input module 110 may transmit the request information, the request sensory information, and the transmission strength information, which are extracted from the user input information, to the sensory pathway determination module 120, the target information extraction module 140, and the substitution signal generation module 150. For example, the input module 110 may transmit the request sensory information and the request information to the sensory pathway determination module 120, transmit the request information to the target information extraction module 140, and transmit the transmission strength information to the substitution signal generation module 150.

The sensory pathway determination module 120 determines a sensory pathway through which the target information is to be transmitted, on the basis of the user input information. The sensory pathway determination module 120 may determine a sensory pathway for each piece of request information. For example, with respect to the request information regarding a “position and distance to a mobile phone,” the sensory pathway determination module 120 may determine a plurality of sensory pathways comprising a sensory pathway through which the visual information is transmitted through goggles worn by the user, and a sensory pathway through which an auditory signal is transmitted through earphones worn by the user, according to a preset table.

The sensory pathway determination module 120 may determine the sensory pathway according to the request sensory information included in the user input information, or may determine the sensory pathway according to the request information included in the user input information. Further, the sensory pathway determination module 120 may determine the sensory pathway according to the target information.

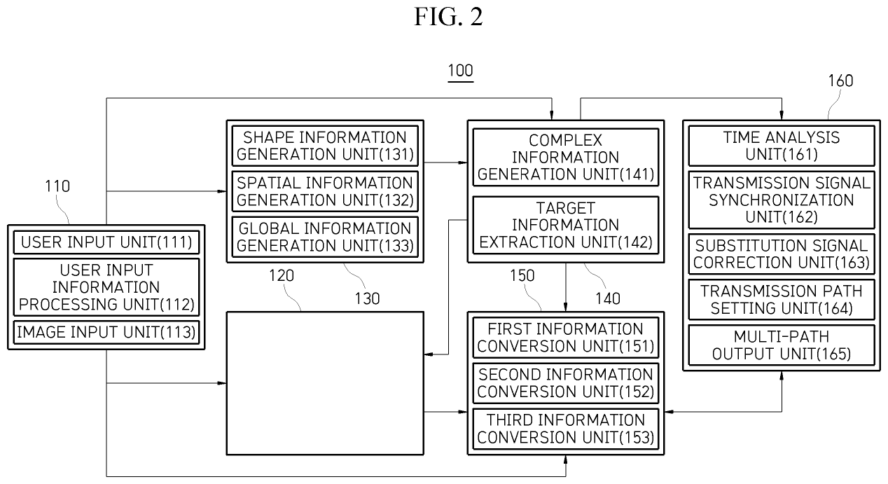

The sensory pathway determination module 120 may determine the sensory pathway for each piece of target information, and the sensory pathway determination module 120 may use the request information or the target information to match the target information and the sensory pathway. A method of determining, by the sensory pathway determination module 120, the sensory pathway will be described below with reference to FIG. 2. The sensory pathway determination module 120 may transmit information on the sensory pathway determined for each piece of target information to the substitution signal generation module 150.

The image information division module 130 divides or analyzes the image information to generate various types of visual information such as shape information, spatial information, global information, and the like, and transmits the generated visual information to the target information extraction module 140. The image information division module 130 may generate visual information corresponding to each frame of the image information, and generate visual information corresponding to a plurality of frames of the image information. For example, the image information division module 130 may generate position information of an animal for each frame of the image information, and generate information on a moving path of an animal, which correspond to 100 frames. The visual information generated by the image information division module 130 may include at least one of shape information, spatial information, and global information, or a combination thereof.

The target information extraction module 140 may generate complex information on the basis of the visual information generated by the image information division module 130. Further, the target information extraction module 140 may extract target information that meets the request information of the user from the visual information and the complex information generated by the image information division module 130.

Hereinafter, in this specification, the term “visual information” is used to collectively refer to shape information, spatial information, global information, and complex information which are generated based on image information.

The target information extraction module 140 extracts the visual information that meets the request information, that is, the target information, from the visual information (including shape information, spatial information, global information, and complex information). That is, the target information extraction module 140 extracts the target information from the visual information on the basis of the request information.

The target information may be a single piece of information or a plurality of pieces of information.

The target information extraction module 140 transmits the target information to the substitution signal generation module 150. Further, the target information extraction module 140 may transmit the target information to the control and output module 160.

The substitution signal generation module 150 converts the target information on the basis of the sensory pathway to generate a substitution signal. The substitution signal generation module 150 converts the visual information extracted by the target information extraction module 140 into a sensory signal corresponding to the visual information, that is, the substitution signal. The substitution signal generation module 150 uses the sensory pathway determined by the sensory pathway determination module 120 in converting the visual information corresponding to the target information into the substitution signal. The substitution signal is a sensory signal that substitutes for the target information, and is a signal generated by the substitution signal generation module 150 to convert the target information into another form of information and transmit the converted information to the sensory organs of the user, and may be at least one sensory signal of a visual signal, an auditory signal, a tactile signal, and a heat sensation signal, or a combination thereof. For example, when the target information is visual information such as a color of a ball, a substitution signal thereof may be a heat sensation signal indicating a predetermined temperature. A method of converting, by the substitution signal generation module 150, the visual information into the substitution signal corresponding thereto may follow a method of numerical conversion or mapping, but the present invention is not limited thereto. The substitution signal generation module 150 converts the target information according to the sensory pathway determined for each piece of target information to generate the substitution signal.

The substitution signal generation module 150 may reflect the transmission strength information included in the user input information to a process of generating the substitution signal. That is, the substitution signal generation module 150 may convert the target information on the basis of the transmission strength information and the sensory pathway to generate the substitution signal. For example, in the case in which the substitution signal generation module 150 converts the target information to generate an auditory signal, the substitution signal generation module 150 may generate a sound wave having a high frequency or amplitude when the transmission strength information is “high.”

The substitution signal generation module 150 transmits the generated substitution signal to the control and output module 160.

The control and output module 160 controls the process of converting the visual information and transmitting the substitution signal to the user. The control and output module 160 may be implemented in the form of a plurality of pieces of hardware. For example, the control and output module 160 may be implemented as a control device and an output device. Further, the control and output module 160 generates control signals necessary to simultaneously transmit individual sensory signals through a plurality of paths. In addition, the control and output module 160 outputs the substitution signals through a single transmission path or a plurality of transmission paths.

The control and output module 160 controls overall operations related to setting the output order of the substitution signals and synchronizing transmission timings. Further, the control and output module 160 transmits the substitution signals to the user. For example, the control and output module 160 transmits an auditory signal, a heat sensation signal, and a vibration signal to earphones, a heater, and a vibration device which is worn by the user so that each of the devices generates an output such as sound, heat, and vibration and transmits the substitution signal to the user. The substitution signal may be a single signal, but may also be a plurality of signals as illustrated.

The control and output module 160 may transmit the target information, which is received together with the substitution signal from the target information extraction module 140, to the user without change. For example, the target information extraction module 140 transmits the target information (e.g., an outline of an animal, and a distance between the user and the animal), which is extracted by the target information extraction module 140 from the visual information generated in an image captured by an infrared camera at night, to the substitution signal generation module 150 and the control and output module 160. The substitution signal generation module 150 converts the target information to generate an auditory substitution signal, and transmits the generated auditory substitution signal to the control and output module 160. The control and output module 160 may output the auditory substitution signal (sound wave) through the earphones worn by the user while simultaneously displaying the target information on the goggles worn by the user.

FIG. 2 is a block diagram illustrating a detailed configuration of the apparatus 100 for sensory substitution and multi-path transmission of visual information according to the embodiment of the present invention.

The input module 110 receives user input information and image information. The input module 110 includes a user input unit 111, a user input information processing unit 112, and an image input unit 113.

The user input unit 111 receives user input information from an external device. The user input information may include at least one of request information, request sensory information, and transmission strength information, or a combination thereof. Specifically, the user input information may include information on a type of an object that the user is interested in, or information (request information) indicating a type of visual information that the user wants to receive from the image information, information (request sensory information) on a sense to receive the request information, information (transmission strength information) expressing a transmission strength of the sense, and the like. For example, the request information may be position information of a specific type of object included in the image information. As a specific example of user input information in the case in which the present invention is applied to virtual reality, the request information may be position information or distance information of another user on the metaverse, and the request sensory information may include vibration and temperature (heat sensation). The transmission strength information may be set to high from among high, medium, and low. The apparatus 100 for sensory substitution and multi-path transmission of visual information may use other senses in addition to the visual sense to maximize the user's experience. For example, when a counterpart user on the metaverse approaches, the user's senses may be amplified due to increased vibration and heat sensation signals.

The user input unit 111 may use a voice or a screen touch to receive the request information. For example, the user designates desired information among a background, a person, an animal, and a product which are displayed in an image through a voice or a screen touch. As a specific example, when the user touches a background part in the image on the screen, the user input unit 111 may recognize that the “background,” which is request information included in the user input information, has been input. When the user touches a person part included in the image on the screen, the user input unit 111 may recognize that the “person,” which is request information included in the user input information, has been input. The request information may be a single piece of information or a plurality of pieces of information. Meanwhile, the user input unit 111 may receive the user input information in a manner in which a preset value is applied through a predetermined button or lever in a special work environment such as a military operation requiring a stealth operation or a noisy environment. For example, when a specific button (e.g., a “person” button, a “position” button, a “distance” button, or a “tactile sense” button) is pressed, the user input unit 111 may regard position information and distance information of the person who shown in the image as request information of the user, and may regard the “tactile sense” as request sensory information input by the user. Further, the user input unit 111 may receive user input information in the form of text. For example, the user input unit 111 may receive user input information including request information and request sensory information in the form of text such as “transmit the distance to the animal by tactile sense.”

The user input information processing unit 112 separately extracts request information, request sensory information, and transmission strength information from the user input information. For example, the user input information processing unit 112 may parse the user input information made in the form of text to obtain the request information, the request sensory information, or the transmission strength information. The user input information processing unit 112 may transmit the request information, the request sensory information, and the transmission strength information that are extracted from the user input information to the sensory pathway determination module 120, the target information extraction module 140, and the substitution signal generation module 150. For example, the user input information processing unit 112 may transmit the request sensory information and the request information to the sensory pathway determination module 120, transmit the request information to the target information extraction module 140, and transmit the transmission strength information to the substitution signal generation module 150.

The image input unit 113 collects image information to be transmitted to the user in the present invention. The image information may include at least one of a still image and a continuous image (video). The image input unit 113 may include an RGB camera, a depth camera (3D depth camera), a time-of-flight (ToF) image sensor, an infrared camera, or the like to collect the image information. Generally, the image information includes various pieces of visual information, and in this specification, for a detailed description, a shape, a border, a position, a distance, a color, texture, a path, a direction, the presence or absence of light, emotion, and overall mood will be described as examples of visual information.

The sensory pathway determination module 120 determines a sensory pathway through which target information is to be transmitted, on the basis of the user input information. The sensory pathway determination module 120 may determine a sensory pathway for each piece of request information. The sensory pathway determination module 120 may determine the sensory pathway according to the request sensory information included in the user input information, or may determine the sensory pathway according to the request information included in the user input information. Further, the sensory pathway determination module 120 may determine the sensory pathway according to the target information. The sensory pathway determination module 120 may determine the sensory pathway for each piece of target information, and the sensory pathway determination module 120 may use the request information or the target information to match the target information and the sensory pathway. The sensory pathway determination module 120 determines the sensory pathway through which the target information is transmitted to the user, on the basis of the user input information. Specifically, the sensory pathway determination module 120 determines whether visual information corresponding to the target information is transmitted with a single signal or a plurality of signals, on the basis of the user input information. The sensory pathway determination module 120 may convert each of a plurality of pieces of target information into a sensory signal corresponding thereto to determine a sensory pathway through which the sensory signal is transmitted to the user, or may convert the same target information into a plurality of sensory signals to determine a sensory pathway through which the plurality of sensory signals are transmitted to the user. For example, when position information of a specific object shown in the image information is target information, the sensory pathway determination module 120 may determine a sensory pathway through which information on left and right or up and down positions of the object are transmitted through sound, or may determine the sensory pathways through which the position information is transmitted through a tactile signal, a vibration, and a heat sensation signal. When determining the sensory pathway on the basis of the user input information, the sensory pathway determination module 120 may determine the sensory pathway on the basis of the request sensory information included in the user input information. Further, the sensory pathway determination module 120 may determine the sensory pathway on the basis of the request information included in the user input information. As an example, when the request sensory information is a “heat sensation,” the sensory pathway determination module 120 may determine a sensory pathway (e.g., a heat sensory pathway through which a temperature is transmitted through a left hand) according to a setting or the user's selection. As another example, when the request information is a “distance to a vehicle,” the sensory pathway determination module 120 may determine a preset sensory pathway (e.g., an auditory sensory pathway through which sound is transmitted through a left ear and a tactile sensory pathway through which a vibration is transmitted through the left hand) for the “distance.” Which information is used to determine the sensory pathway is determined by the user's selection or setting.

The sensory pathway determination module 120 may transmit information on the sensory pathway determined for each piece of target information to the substitution signal generation module 150.

The image information division module 130 divides or analyzes the image information to generate various types of visual information such as shape information, spatial information, global information, and the like, and transmits the generated visual information to the target information extraction module 140. The image information division module 130 may generate visual information corresponding to each frame of the image information, and may also generate visual information corresponding to a plurality of frames of the image information. For example, the image information division module 130 may generate position information of an animal for each frame of the image information, and may also generate information on a moving path of the animal, which correspond to 100 frames. The image information division module 130 may divide the image information step by step to extract various pieces of visual information included in the frames of the image or video. The visual information generated by the image information division module 130 includes raw information such as coordinates of pixels constituting a frame and information obtained through image processing such as a channel of the frame. Further, the visual information may include a significant analysis result obtained by using an inference technique using an algorithm such as deep learning-based object detection, region segmentation, and relational information analysis. Therefore, the image information division module 130 may perform division and analysis in the order of preferentially dividing the shape information that can be extracted from the frame of the image or video with a simple operation, then dividing the spatial information that relatively requires more user input or additional analysis work, and finally, generating the global information or the complex information. The image information division module 130 may include a shape information generation unit 131, a spatial information generation unit 132, and a global information generation unit 133, wherein the shape information generation unit 131 may divide the image information to generate shape information, the spatial information generation unit 132 may divide the image information to generate spatial information, and the global information generation unit 133 may analyze the image information to generate global information or complex information. Examples of the shape information, the spatial information, the global information, and the complex information are shown in Table 1.

TABLE 1

| Visual information | Examples |
|---|---|
| Shape information | Shape, outline (border), color, presence or |
| | absence of light, or texture |
| Spatial information | Position, distance, direction, or path |
| Global information | Color or shape extracted from entire image area |
| Complex information | Path (generated based on position information), |
| | emotion, or mood |

The target information extraction module 140 may generate complex information on the basis of the visual information generated by the image information division module 130. Further, the target information extraction module 140 may extract the target information that meets the request information of the user from the visual information and the complex information generated by the image information division module 130. For example, when the request information is an “outline of an animal,” the target information extraction module 140 may extract, as the target information, visual information showing an outline of a dog or a cat from the visual information.

As described above, the target information extraction module 140 receives the visual information (e.g., shape information, spatial information, and global information) generated by the image information division module 130, on the basis of the image information, and in this case, the target information extraction module 140 may use the request information as a reference to selectively receive necessary visual information.

The target information extraction module 140 transmits the target information to the substitution signal generation module 150. The target information extraction module 140 extracts the visual information that meets the request information, that is, the target information, from the visual information such as the shape information, the spatial information, the global information, the complex information, and the like. That is, the target information extraction module 140 extracts the target information that meets the request information from the visual information (shape information, spatial information, and global information) that is generated by the image information division module 130 and the visual information (complex information) that is generated by the target information extraction module 140. The target information extraction module 140 includes a complex information generation unit 141 and a target information extraction unit 142.

The complex information generation unit 141 generates complex information on the basis of the visual information generated by the image information division module 130. For example, the complex information generation unit 141 may obtain mood information on the basis of shape information such as a shape of an object and a border (outline) and global information such as a color extracted from an entire image area. The complex information generation unit 141 may generate the complex information on the basis of a plurality of pieces of shape information or a plurality of pieces of spatial information and generate complex information such as emotion or mood information on the basis of overall image information (global information), and artificial intelligence techniques may be used in the process of generating the complex information.

The target information extraction unit 142 extracts the target information that meets the request information from the visual information (shape information, spatial information, global information, and complex information).

The substitution signal generation module 150 converts target information on the basis of a sensory pathway to generate a substitution signal. The substitution signal generation module 150 converts the target information extracted by the target information extraction module 140 into a sensory signal that substitutes for the target information, that is, a substitution signal. The substitution signal generation module 150 converts the target information extracted by the target information extraction module 140 into a substitution signal corresponding to the target information. The substitution signal generation module 150 determines, based on reference data such as a setting table, which substitution signal (sound wave) that meets the target information is transmitted through the sensory pathway (e.g., distance between the vehicle and the user is transmitted as an auditory sensory pathway through a left ear) for each piece of target information, and converts the target information to generate a substitution signal. That is, the substitution signal generation module 150 converts the target information according to the sensory pathway determined for each piece of target information and generates a substitution signal corresponding thereto.

Meanwhile, the substitution signal generation module 150 may reflect time information (including time T_{c} for information conversion, time Tₜ for transmission, time T_{d} for delay, and time Tₚ for perception) calculated by the control and output module 160 to the process of generating the substitution signal. For example, when it is determined that a time required for transmission is insufficient according to a result of time analysis of the control and output module 160, the substitution signal generation module 150 may simplify the process of converting the sensory signal or the result of the conversion to shorten the time T_{c} for information conversion or the time Tₜ for transmission. When the time T_{c} for information conversion or the time Tₜ for transmission cannot be shortened by the substitution signal generation module 150, a transmission signal synchronization unit 162 of the control and output module 160 delays a synchronization time (time point) to secure the transmission time as much as the time required for transmission.

In the present embodiment, the substitution signal generation module 150 may include a first information conversion unit 151 which converts target information into an auditory signal (sound), a second information conversion unit 152 which converts target information into a tactile signal (vibration), and a third information conversion unit 153 which converts target information into a heat sensation signal (temperature). However, a component may be added to the substitution signal generation module 150 according to the addition of a convertible sense (e.g., smell) or subdivision of the sense (e.g., subdividing a tactile signal into a vibration, a haptic sensation, and a pressure).

The first information conversion unit 151 converts target information into an auditory signal (sound wave). The first information conversion unit 151 mainly converts shape information such as a shape, a position, the presence or absence of light, a color, a direction, and the like or spatial information into a sound wave. As a specific example, the first information conversion unit 151 may define a frequency in advance for each pixel or pixel group at a vertical position of an image to generate a sound wave so that a difference in a vertical position of an object is distinguished by a frequency. That is, the first information conversion unit 151 may map the vertical position of the image to a frequency of a sound wave. Further, the first information conversion unit 151 may generate a sound wave by mapping a horizontal position of the image to reproduction time information of a sound or stereo signal and mapping a brightness value of the pixel corresponding to the shape or border of the object to an amplitude. Further, when the spatial information is expressed as a sound, the first information conversion unit 151 may complexly combine the frequency and the amplitude to generate a sound wave with an increased reproduction frequency and volume as the distance to the object is reduced, and to generate a sound wave with a reduced reproduction frequency and volume as the distance to the object is increased. When the visual information is color information (e.g., color of an object, color extracted from the entire image area, emotion or mood on the basis of global information, etc.), it is possible to analyze a distribution of the color using deep learning technology, and thus the first information conversion unit 151 may convert the visual information in a manner of being expressed as a light sound wave in the case of the analyzed information being a bright feeling or mood and expressed as a sound wave of a slow or low scale in the case of the analyzed information being a dark or depressing mood or emotion.

The second information conversion unit 152 converts target information into a tactile signal. The second information conversion unit 152 mainly converts target information such as a distance, texture, a position, a path, a direction, or the like into a tactile signal. In the apparatus 100 for sensory substitution and multi-path transmission of visual information, a device to which a tactile signal is finally transmitted may be a vibration device attached to a wrist or worn on the body. When a vibration generating element is positioned in upper, lower, left, and right positions of the vibration device attached to the wrist, the second information conversion unit 152 may generate tactile signals for a plurality of channels so that only the element corresponding to a specific path or direction generates a vibration. The control and output module 160 may transmit the direction or path of the object to the user by transmitting a control signal to the vibration device on the basis of the tactile signals generated in this way so that the element that meets the path or direction generates vibration.

Meanwhile, when the target information is distance information, the second information conversion unit 152 may convert distance information into a tactile signal in a manner of changing a period or strength of a vibration according to a distance.

The third information conversion unit 153 converts target information into a heat sensation signal. The third information conversion unit 153 may mainly convert target information related to color, emotion, or mood into a heat sensation signal. In the apparatus 100 for sensory substitution and multi-path transmission of visual information, a device to which a heat sensation signal is finally transmitted may be a heat transmission device (heater). As a specific example, the third information conversion unit 153 may convert target information on blue or dark mood into a heat sensation signal having a low temperature in a set temperature range in which the heat transmission device is transmitted, and may convert the target information on red or hot mood into a heat sensation signal having a high temperature in the set temperature range, on the basis of a temperature range that can be transmitted by the heat transmission device.

Each of the information conversion units 151, 152, and 153 of the substitution signal generation module 150 may convert the target information on the basis of transmission strength information and a sensory pathway to generate a substitution signal. For example, when the first information conversion unit 151 converts the target information to generate an auditory signal, the first information conversion unit 151 may reflect the fact that the transmission strength information included in the user input information is “high” to generate a sound wave having an increased frequency or amplitude.

In order to efficiently transmit the target information to the user, the substitution signal generation module 150 may convert a plurality of pieces of target information individually into different substitution signals, and may convert the same visual information into a plurality of substitution signals. In this case, the substitution signal generation module 150 may derive a final conversion result by combining the conversion results of the plurality of information conversion units 151, 152, and 153. For example, even when a single piece of position information is converted, the substitution signal generation module 150 may convert the position information into a sound wave representing left and right through the first information conversion unit 151, and convert the position information into a vibration signal to be transmitted to a left-side element of the vibration device worn on the wrist of the left hand through the second information conversion unit 152. In this case, the control and output module 160 may simultaneously output the sound wave and the vibration signal related to the position information and may allow the user to more efficiently perceive the position information.

The substitution signal generation module 150 transmits the generated substitution signal to the control and output module 160.

The control and output module 160 controls overall operations related to setting the output order of the substitution signals and synchronizing the transmission timings. The control and output module 160 transmits the substitution signal generated by the substitution signal generation module 150 to the user. The control and output module 160 controls and manages the flow of transmission signals necessary for overall operations for multi-path transmission of target information. Further, the control and output module 160 may output the substitution signal through multiple paths.

Meanwhile, the control and output module 160 may be physically divided into separate devices. For example, the control and output module 160 may include a control device and an output device.

The control and output module 160 includes a transmission path setting unit 164, and a multi-path output unit 165. The control and output module 160 may further include a time analysis unit 161 and a transmission signal synchronization unit 162 for synchronizing a substitution signal, and may further include a substitution signal correction unit 163 for correcting the substitution signal. The substitution signal synchronization and correction of the control and output module 160 is necessary to effectively transmit the target information to the user.

The control and output module 160 synchronizes a plurality of substitution signals for each piece of target information on the basis of the sensory pathway for each piece of target information determined by the sensory pathway determination module 120 and transmits the plurality of synchronized substitution signals to the user. Further, the control and output module 160 corrects the characteristic (e.g., strength) or type of the substitution signal according to the image information and the user's action (user's reaction). The control and output module 160 may recognize the user's action (user's reaction) through analysis of the image information.

First, functions of the time analysis unit 161 and the transmission signal synchronization unit 162 in charge of synchronizing the substitution signals will be described. The time analysis unit 161 provides time information which is necessary for the transmission signal synchronization unit 162 to determine a synchronization time point to the transmission signal synchronization unit 162. The time analysis unit 161 calculates \textcircled{1} a time T_{c} for information conversion for each sense, \textcircled{2} a time Tₜ for transmission for each sense, \textcircled{3} a time T_{d} for delay of a transmission device, and \textcircled{4} a time Tₚ for perception for each sensory organ, on the basis of a sensory pathway for each piece of target information, and transmits information on the calculated times T_{c}, Tₜ, T_{d}, and Tₚ to the transmission signal synchronization unit 162. In Table 2, the respective times calculated by the time analysis unit 161 are described.

TABLE 2

| Type | Symbol | Descriptions |
|---|---|---|
| Time for | T_{c} | Time required for converting target information |
| conversion | | into substitution signal for each sense |
| Time for | Tₜ | Time required to transmitting or reproducing |
| transmission | | information for each sense |
| Time for | T_{d} | Time for delay of device that transmits |
| delay | | information to each sensory organ |
| Time for | Tₚ | Time required for target sensory receptor to |
| perception | | perceive transmission signal |

The time analysis unit 161 may calculate the time T_{c} for information conversion for each sense on the basis of the time (time required for generating the substitution signal) required for each of the conversion units 151, 152, and 153 of the substitution signal generation module 150 to convert the target information into the substitution signal. Further, the time Tₜ for transmission for each sense is a time required for transmitting information to each target sensory organ. The time Tₜ for transmission for each sense may be different for each sense. For example, a different length of time may be required for each sense, such as 0.5 seconds for an auditory sense, 0.8 seconds for a tactile sense, 1 second for a heat sensation, or the like. Further, the time T_{d} for delay of the transmission device may be a time for transmission of the transmission device, and may vary according to the characteristics of the device. Therefore, the time analysis unit 161 may calculate the time T_{d} for delay by reflecting the information disclosed by the device manufacturer. Further, the time analysis unit 161 may calculate the time Tₚ for perception for each sensory organ on the basis of a general known value (result of a previous study). However, there may be a difference in perception time for each user, and since some people do not feel a specific sense, the substitution signal correction unit 163 corrects the substitution signal according to the user's action or user's reaction.

The transmission signal synchronization unit 162 determines a synchronization time point for a plurality of substitution signals to be transmitted to the user on the basis of the frame of the visual information extracted by the target information extraction module 140 and information on the time calculated by the time analysis unit 161, and performs a synchronization operation. In order for the transmission signal synchronization unit 162 to synchronize the plurality of substitution signals, a time T_{c} (time for conversion) required for converting the target information into a substitution signal for each target sense, a time Tₜ (time for transmission) required for transmitting or reproducing information for each sense, a time T_{d} (time for delay) for delay of the device required for transmitting information to each sensory organ, and a time Tₚ (time for perception) that the target sensory receptor perceives the transmission signal should be considered. The reason that such temporal consideration is necessary for synchronization is that, when various pieces of visual information of a video or image are transmitted with a plurality of substitution signals, there is a limit to the strength of the signal and amount of information that can be transmitted to each sensory organ, and the recognition of the same signal is different depending on the user. For example, a substitution signal in the form of a sound wave requires reproduction time due to its characteristics. Meanwhile, a substitution signal in the form of a vibration signal may be shorter or longer than the reproduction time of the sound wave. Therefore, when substitution signals obtained by converting different pieces of target information derived from the same image frame are transmitted through a plurality of sensory pathways, the control and output module 160 should synchronize the substitution signals for the same frame and transmit the synchronized substitution signals to the user at the same time.

As another example, even when the same visual information is transmitted to the user through a plurality of sensory pathways, since the amount of information that can be transmitted is different for each sensory organ, the pieces of transmitted information should be synchronized. A synchronization time point Tₛ at which the converted substitution signals are transmitted through a single pathway or a plurality of pathways may vary according to the request information or the target information. The transmission signal synchronization unit 162 may determine a time required for the longest time as the synchronization time point as shown in Equation 1 in consideration of the time T_{c} for information conversion, the sum (Tₜ+T_{d}) of the time Tₜ for transmission and the time T_{d} for delay, and the time Tₚ for perception for each sensory organ for the target information extracted by the target information extraction module 140.

Tₛ=max(T_{c},Tₜ+T_{d},Tₚ) [Equation 1]

Next, the substitution signal correction unit 163 that performs the correction of the substitution signal will be described as follows. In spite of a difference between an attachment position of a camera for inputting the image information and a position of a device to which the converted substitution signals are transmitted, when the substitution signals are transmitted to the user without correction, it is difficult for the user to accurately perceive the position or distance to the object. Therefore, the substitution signal correction unit 163 performs a function of correcting the substitution signals by resolving the discrepancy. For example, generally, the camera for inputting the image information is attached to glasses, a necklace, or a chest using a fixing device, whereas location information or depth information is transmitted through sound or to a vibration device which is attached to the hand. In this case, since the position/distance viewed from the head or chest and the position of the ear or hand receiving the substitution signals are relatively different, the position information or the distance information is not correspondingly transmitted to a sound device or a tactile device. Therefore, a function of correcting a difference between a position at which the image information is input and a position at which the substitution signal is transmitted so that the user can efficiently perceive the difference is essential, and the substitution signal correction unit 163 performs such a function.

Further, the substitution signal correction unit 163 corrects the characteristic (e.g., strength) or type of the substitution signal according to the image information, the user's action, or the user's reaction. The substitution signal correction unit 163 may analyze the image information to recognize the user's action (or user's reaction). The substitution signal correction unit 163 may determine the user's sensitivity on the basis of the image information and the user's action, and correct the characteristic or type of the substitution signal on the basis of the user's sensitivity. Specifically, when the visual information is converted into an auditory sense, a tactile sense, a heat sensation, or the like and transmitted, the sensitivity to a specific sense may be different depending on the user. Therefore, when it is determined that the user's sensitivity to the substitution signal is low, the substitution signal correction unit 163 increases the strength of the substitution signal or transitions the sensory pathway for the corresponding target information to another sensory pathway (e.g., converting an auditory sense into a heat sensation). As an example of increasing the strength of the substitution signal, there may be an increase in size of the auditory signal.

The control and output module 160 sets a single pathway or a plurality of pathways with reference to a result of the synchronization of the transmission signal synchronization unit 162, and outputs the substitution signal to the device attached to the user through the set transmission path. The transmission path setting unit 164 receives the substitution signal that has been converted by the substitution signal generation module 150, and sets the transmission path of the substitution signal on the basis of the sensory pathway for each piece of target information in consideration of the time T_{c} for information conversion for each pathway, the time Tₜ for transmission for each sense, the time T_{d} for delay for each sense of the transmission device, and the time Tₚ for perception for each sense. The control and output module 160 may transmit the individual substitution signal to the user according to the synchronization time point derived by the transmission signal synchronization unit 162 on the basis of the four types of times (\textcircled{1} time T_{c} for information conversion for each sense, \textcircled{2} time Tₜ for transmission for each sense, \textcircled{3} the T_{d} for delay of the transmission device, and \textcircled{4} time Tₚ for perception for each sensory organ) calculated by the time analysis unit 161, and correct the transmission path and intensity (strength) of the substitution signal in consideration of the user's action or the user's reaction to individual senses. Since the visual information is information on a sense having the largest amount of information among the five senses, when the visual information is converted into another sensory information, information abbreviation occurs. Therefore, there is a problem in that the identical substitution signal may be generated through the conversion of non-identical visual information, and this problem may be solved through personalization correction on the basis of the user's action. The multi-path output unit 165 simultaneously transmits individual substitution signals included in a predetermined temporal range through the transmission path set by the transmission path setting unit 164 with reference to the synchronization signals generated by the transmission signal synchronization unit 162.

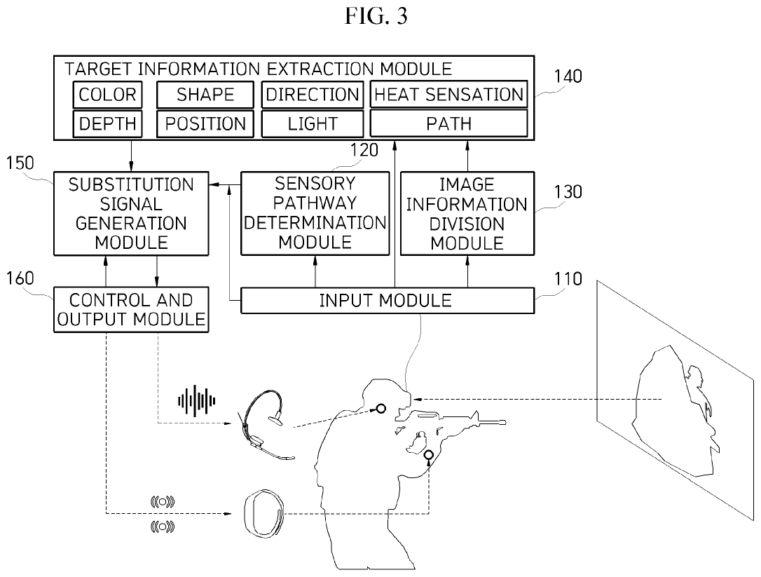

FIG. 3 is an exemplary diagram illustrating a concept of sensory substitution and multi-path transmission of visual information according to the present invention. FIG. 3 illustrates a situation in which target information is converted into a substitution signal when performing a military operation among various application examples of the present invention and is transmitted to a user through multiple paths.

The input module 110 receives user input information and image information (still image or video), and in this case, request information included in the user input information is information that meets the purpose of the user performing reconnaissance or military operations, and may be a position of a person, a distance to the person, a location of a vehicle, a distance to the vehicle, and the like. The input module 110 parses the user input information to extract request information, request sensory information, and transmission strength information. For example, the request information may be a “distance to a vehicle,” the request sensory information may be a “heat sensation,” and the transmission strength information may be “high.”

The sensory pathway determination module 120 determines a sensory pathway on the basis of the user input information. The sensory pathway determination module 120 may determine the sensory pathway on the basis of the request sensory information, and determine the sensory pathway on the basis of the request information. For example, when the request sensory information is a “heat sensation,” the sensory pathway determination module 120 may determine a corresponding sensory pathway (e.g., a heat sensory pathway through which temperature is transmitted through a left hand). As another example, when the request information is a “distance to a vehicle,” the sensory pathway determination module 120 may determine a preset sensory pathway (e.g., an auditory sensory pathway through which a sound is transmitted through a left ear and a tactile sensory pathway through which a vibration is transmitted through a left hand) for the “distance.” Which information is used to determine the sensory pathway is determined by the setting.

The image information division module 130 divides the image information to generate various pieces of visual information. For example, the image information division module 130 divides the image information to generate shape information such as a shape of an object, a border of the object, an outline of a background, a color of the object, a color of the background, the presence or absence of light, texture of the object, or the like, spatial information such as a location of an object, a distance between the user and the object, a distance between the object and another object, a direction of movement of the object, or the like, and global information such as a dominant color or shape of the entire image, or the like.

The target information extraction module 140 extracts target information, which is visual information that meets the request information, on the basis of the visual information generated by the image information division module 130. For example, the target information extraction module 140 extracts information on a location of the vehicle and information on a distance between the user and the vehicle, which are the target information, that meet the location of the vehicle and the distance to the vehicle, which are the request information.

The substitution signal generation module 150 converts the target information to generate a substitution signal with reference to transmission strength information included in the user input information. For example, the substitution signal generation module 150 converts the information on the distance to the vehicle to generate an auditory signal and vibration signal having a transmission strength of “high.”

The control and output module 160 may transmit the substitution signal through multiple paths to the user to allow the user to efficiently perceive necessary information. For example, the control and output module 160 may simultaneously output the auditory signal and the vibration signal through the earphones worn by the user and the vibration device to allow the user to perceive the information on the distance to the vehicle.

In the method for sensory substitution and multi-path transmission according to the present invention, unlike in the conventional sensory substitution technology, it is possible to perform synchronized transmission of complex visual information and improve the perception of the user. As an example, as illustrated in FIG. 3, information on a red ball near the left of a soldier with a gun is complex information that cannot be transmitted in the conventional sensory substitution technology or devices. According to the method for sensory substitution and multi-path transmission according to the present invention, shape information (round shape of the ball) may be converted into a sound, position information (left side of the soldier) may be transmitted to a left vibration element of the vibration device attached to the wrist, and at the same time, distance information (distance between the soldier and an object in front of the soldier) may be transmitted at a vibration period or with a vibration intensity of the vibrating device, and a heat sensation signal (temperature information) set to a high level, such as color information (red), may be simultaneously transmitted through the heat transmission device.

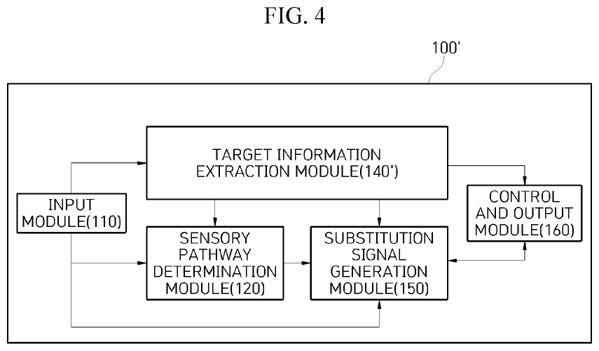

FIG. 4 is a block diagram illustrating a configuration of an apparatus 100′ for sensory substitution and multi-path transmission of visual information according to another embodiment of the present invention.

The apparatus 100′ for sensory substitution and multi-path transmission of visual information according to another embodiment of the present invention includes an input module 110, a sensory pathway determination module 120, a target information extraction module 140′, a substitution signal generation module 150, and a control and output module 160.

The embodiment of FIG. 4 is different from the embodiment of FIG. 1 in terms of device configuration. The embodiment of FIG. 4 includes a single target information extraction module 140′ in which the functions of the image information division module 130 and the target information extraction module 140 of the embodiment of FIG. 1 are integrated. The embodiment of FIG. 4 may be applied to a case in which parallel processing of an image information division operation and a target information extraction operation is not required because a workload is not large, as compared to the embodiment of FIG. 1.

The target information extraction module 140′ may divide image information received from the input module 110, generate various pieces of visual information such as shape information, spatial information, global information, and the like, generate complex information on the basis of the visual information, extracts target information that meets request information received from the sensory pathway determination module 120 from the generated shape information, spatial information, global information, and complex information, and transmits the extracted target information to the substitution signal generation module 150. Further, the target information extraction module 140′ may transmit the target information to the control and output module 160. The target information extraction module 140′ includes a shape information generation unit 131, a spatial information generation unit 132, a global information generation unit 133, a complex information generation unit 141, and a target information extraction unit 142 in order to perform the above-described functions, and the function of each component of the target information extraction module 140′ is the same as that described above with reference to FIG. 2.

Meanwhile, since the functions of the input module 110, the sensory pathway determination module 120, the substitution signal generation module 150, and the control and output module 160 which are included in the apparatus 100′ for sensory substitution and multi-path transmission of visual information are the same as those described above with reference to FIGS. 1 to 3, descriptions thereof will be omitted.

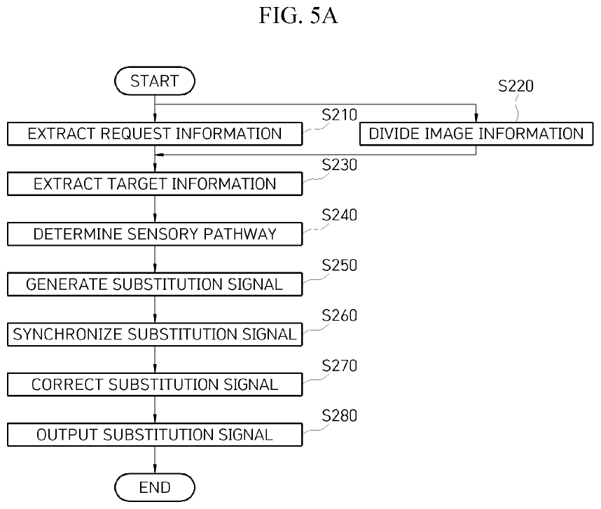

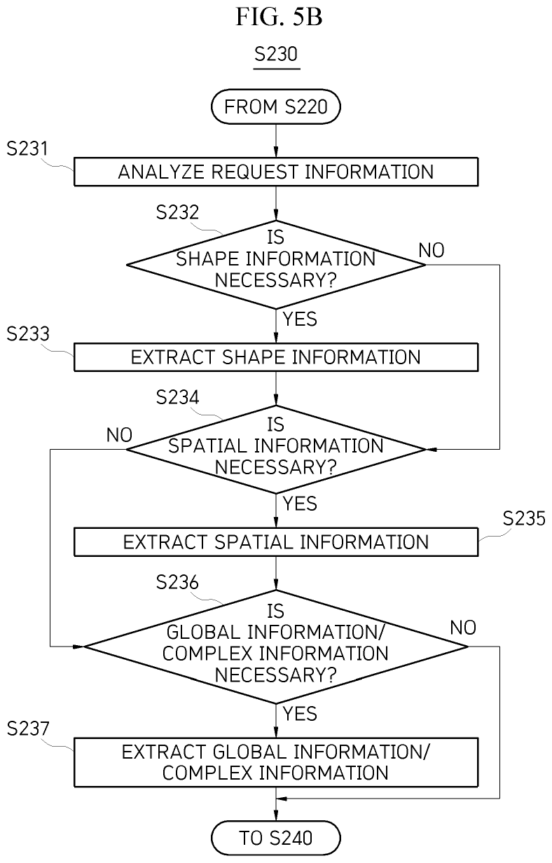

FIGS. 5A and 5B illustrate flowcharts for describing a method for sensory substitution and multi-path transmission of visual information according to an embodiment of the present invention.

The method for sensory substitution and multi-path transmission of visual information according to the present invention includes operations S210 to S280.

Operation S210 is an operation of extracting request information. The apparatuses 100 and 100′ for sensory substitution and multi-path transmission of visual information receive user input information, and the user input information may include request information (information indicating a type of visual information that the user wants to receive), request sensory information (information on a sense that is a medium for transmitting the request information), transmission strength information, and the like. The apparatuses 100 and 100′ for sensory substitution and multi-path transmission of visual information may extract the request information from the user input information.

Operation S220 is an operation (operation of generating visual information) of dividing image information. The apparatuses 100 and 100′ for sensory substitution and multi-path transmission of visual information collect the image information from an external device. The apparatuses 100 and 100′ for sensory substitution and multi-path transmission of visual information may use an RGB camera, a depth camera (3D depth camera), a ToF image sensor, an infrared camera, or the like in order to collect the image information. The image information may include at least one of a still image and a continuous image (video). The apparatuses 100 and 100′ for sensory substitution and multi-path transmission of visual information divide the image information to generate visual information such as shape information, spatial information, global information, and the like, and generate complex information on the basis of the generated visual information. As described above, in the present invention, the information which is generated based on the image information, such as the shape information, the spatial information, the global information, the complex information, and the like, is collectively referred to as “visual information.”

Operation S230 is an operation of extracting target information. The apparatuses 100 and 100′ for sensory substitution and multi-path transmission of visual information determine whether each piece of visual information which is generated in operation S220 by analyzing the request information is necessary, and extract visual information that meets the request information, that is, target information. Since necessary information may appear complexly according to the request information, the apparatuses 100 and 100′ for sensory substitution and multi-path transmission of visual information first extract necessary shape information according to the request information, then extract spatial information corresponding to the request information, analyze a correlation between the request information and the global information/complex information, and extract the global information or the complex information as necessary. In addition, the apparatuses 100 and 100′ for sensory substitution and multi-path transmission of visual information set the extracted visual information (shape information, spatial information, global information, and complex information) as the target information.

Operation S230 will be described below in detail with reference to FIG. 5B. Operation S230 includes operations S231 to S237.

Operation S231 is an operation of analyzing the request information. The apparatuses 100 and 100′ for sensory substitution and multi-path transmission of visual information analyze the request information and specify necessary information. For example, in the case in which the present invention is applied to a survival game in virtual reality, when the request information is determined to be a “person” on the basis of a user input (screen or button touch), the apparatuses 100 and 100′ for sensory substitution and multi-path transmission of visual information may derive a shape, an outline (border), a position, a distance, a path, etc. as the visual information related to the request information. Referring to Table 1 based on such a result, the apparatuses 100 and 100′ for sensory substitution and multi-path transmission of visual information may determine that the shape information (shape or outline), the spatial information (position or distance), and the complex information (path) are necessary in subsequent operations (operations S232, S234, and S236).

Operation S232 is an operation of determining whether the shape information is necessary. The apparatuses 100 and 100′ for sensory substitution and multi-path transmission of visual information perform operation S233 when it is determined that the shape information is necessary as a result of analyzing the request information, and otherwise, perform operation S234.

Operation S233 is an operation of extracting the shape information. The apparatuses 100 and 100′ for sensory substitution and multi-path transmission of visual information extract shape information that meets the request information from the visual information generated in operation S220, and then proceed to operation S234.

Operation S234 is an operation of determining whether the spatial information is necessary. The apparatuses 100 and 100′ for sensory substitution and multi-path transmission of visual information perform operation S235 when it is determined that the spatial information is necessary as a result of analyzing the target information, and otherwise, perform operation S236.

Operation S235 is an operation of extracting the spatial information. The apparatuses 100 and 100′ for sensory substitution and multi-path transmission of visual information extract the spatial information that meets the request information from the visual information generated in operation S220, and then proceed to operation S236.

Operation S236 is an operation of determining whether the global information or the complex information is necessary. The apparatuses 100 and 100′ for sensory substitution and multi-path transmission of visual information proceed to operation S237 when it is determined that the global information or the complex information is necessary as a result of analyzing the target information, and otherwise, proceed to operation S240.

Operation S237 is an operation of extracting the global information or the complex information. The apparatuses 100 and 100′ for sensory substitution and multi-path transmission of visual information extract the global information or the complex information that meets the request information from the visual information generated in operation S220, and then proceed to S240.

Referring to FIG. 5A again, subsequent operations including operation S240 will be described as follows.

Operation S240 is an operation of determining a sensory pathway. The apparatuses 100 and 100′ for sensory substitution and multi-path transmission of visual information may determine a sensory pathway according to the request sensory information included in the user input information, or may determine a sensory pathway according to the request information included in the user input information. Further, the apparatuses 100 and 100′ for sensory substitution and multi-path transmission of visual information may determine a sensory pathway according to the target information.

The apparatuses 100 and 100′ for sensory substitution and multi-path transmission of visual information may determine a sensory pathway for each piece of target information, and the apparatuses 100 and 100′ for sensory substitution and multi-path transmission of visual information may use the request information or the target information in order to match the target information and the sensory pathway.

Operation S250 is an operation of converting the target information and generating a substitution signal. The apparatuses 100 and 100′ for sensory substitution and multi-path transmission of visual information convert the target information extracted in operation S230 into a substitution signal corresponding thereto. The substitution signal is a signal for transmitting the target information to a sensory organ of the user, and may include a visual signal, an auditory signal, a tactile signal, or a heat sensation signal. The apparatuses 100 and 100′ for sensory substitution and multi-path transmission of visual information may convert a single piece of target information into several substitution signals, and convert a plurality of pieces of target information into individual substitution signals corresponding to each piece of target information. For example, one piece of target information may be converted into a plurality of substitution signals such as a sound wave, a tactile signal, a heat sensation signal, and the like, and three pieces of target information may be converted into a sound wave, a tactile signal, and a heat sensation signal, respectively.

Operation S260 is an operation of synchronizing the substitution signals. The apparatuses 100 and 100′ for sensory substitution and multi-path transmission of visual information synchronize the plurality of substitution signals generated in operation S250 on the basis of the target information and the sensory pathway for each piece of target information. The apparatuses 100 and 100′ for sensory substitution and multi-path transmission of visual information calculate \textcircled{1} a time T_{c} for information conversion for each sense, \textcircled{2} a time Tₜ for transmission for each sense, \textcircled{3} a time T_{d} for delay of a transmission device, and \textcircled{4} a time Tₚ for perception for each sensory organ as each piece of target information, on the basis of the sensory pathway for each piece of target information, and perform synchronization on the plurality of substitution signals on the basis of the information on the calculated times \textcircled{1} to \textcircled{4}. In determining a synchronization time point, Equation 1 described above may be used.

Operation S270 is an operation of correcting the substitution signal. The apparatuses 100 and 100′ for sensory substitution and multi-path transmission of visual information correct the characteristic (e.g., strength) or type of the substitution signal according to the image information and the user's action. The apparatuses 100 and 100′ for sensory substitution and multi-path transmission of visual information may recognize the user's action through analysis of the image information. Further, the apparatuses 100 and 100′ for sensory substitution and multi-path transmission of visual information may correct the substitution signal on the basis of a position of an image information input device and a position of a substitution signal transmission device. For example, when a camera into which the image information is input is on a necklace, the target information is position information and distance information of the object, and the position information is transmitted to the user through a vibration device attached to a hand, the apparatuses 100 and 100′ for sensory substitution and multi-path transmission of visual information correct a tactile signal (vibration signal) transmitted to the vibration device on the basis of a difference between a position of the camera and a position of the vibration device. In this case, each of the position of the camera and the position of the vibration device may be expressed as 3D coordinates and the difference between the two positions may be expressed as a 3D vector.

Operation S280 is an operation of outputting the substitution signals. The apparatuses 100 and 100′ for sensory substitution and multi-path transmission of visual information set a single pathway or a plurality of pathways for each substitution signal with reference to the synchronization time point of the substitution signals, and output the substitution signals to the device attached to the user through the set transmission path. The apparatuses 100 and 100′ for sensory substitution and multi-path transmission of visual information may transmit a single piece of information to the user through a plurality of transmission paths, and may transmit a plurality of pieces of information to the user through different separate paths. The apparatus 100 for sensory substitution and multi-path transmission of visual information outputs the substitution signals according to the synchronization signals.

Meanwhile, in the description with reference to FIGS. 5A and 5B, each operation may be further divided into additional operations or may be combined into fewer operations according to the embodiments of the present invention. Further, some operations may be omitted as necessary, and the order of the operations may be changed. In addition, the contents of FIGS. 1 to 4 may be applied to the contents of FIGS. 5A and 5B even when other contents are omitted. Further, the contents of FIGS. 5A and 5B may be applied to the contents of FIGS. 1 to 4.

The above-described method for sensory substitution and multi-path transmission of visual information has been described with reference to the flowcharts shown in the drawings. For simplicity, the method has been shown and described as a series of blocks, the present invention is not limited to the order of the blocks, some blocks may be performed in the order different from that shown and described herein with other blocks or performed in concurrently with other blocks, and various other branches, flow paths, and orders of blocks may be implemented to achieve the same or similar result. Further, not all illustrated blocks may be required for implementation of the method described herein.

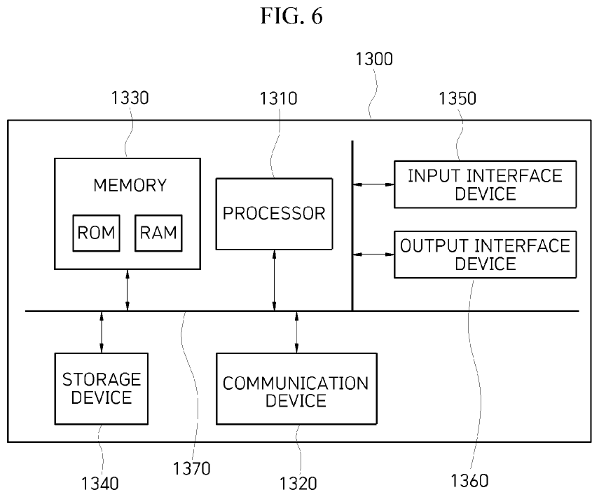

FIG. 6 is a block diagram illustrating a computer system for implementing the method according to the embodiment of the present invention.

Referring to FIG. 6, a computer system 1300 may include at least one of a processor 1310, a memory 1330, an input interface device 1350, an output interface device 1360, and a storage device 1340 which communicate via a bus 1370. The computer system 1300 may further include a communication device 1320 coupled to a network. The processor 1310 may be a central processing unit (CPU) or a semiconductor device that executes instructions stored in the memory 1330 or the storage device 1340. The memory 1330 and the storage device 1340 may include various types of volatile or non-volatile storage media. For example, the memory may include a read only memory (ROM) and a random-access memory (RAM). In the embodiment of the present invention, the memory may be positioned inside or outside the processor, and the memory may be connected to the processor through various known units. The memory may include various types of volatile or non-volatile storage media. For example, the memory may include a ROM or a RAM.

Therefore, the embodiment of the present invention may be implemented as a method implemented in a computer or as a non-transitory computer-readable medium having computer-executable instructions stored therein. In an embodiment, when the computer-executable instructions are executed by the processor, the computer-executable instructions may perform the method according to at least one aspect of the present invention.

The communication device 1320 may transmit or receive a wired signal or a wireless signal.

Further, the method according to the embodiment of the present invention may be implemented in the form of program instructions that can be executed through various computer units and recorded on computer readable media.

The computer readable media may include program instructions, data files, data structures, or combinations thereof. The program instructions recorded on the computer readable media may be specially designed and prepared for the embodiments of the invention or may be available well-known instructions for those skilled in the field of computer software. The computer readable media may include a hardware device configured to store and execute program instructions. Examples of the computer readable media include magnetic media such as a hard disk, a floppy disk, and a magnetic tape, optical media such as a compact disc read only memory (CD-ROM) and a digital video disc (DVD), magneto-optical media such as a floptical disk, and a hardware device, such as a ROM, a RAM, or a flash memory, that is specially made to store and perform the program instructions. Examples of the program instruction include machine code generated by a compiler and high-level language code that can be executed in a computer using an interpreter and the like.

While embodiments of the present invention have been described above in detail, the scope of the present invention is not limited thereto, but encompasses several modifications and improvements by those skilled in the art using basic concepts of embodiments of the present invention defined by the appended claims.

For reference, terms described in the specification such as “unit” or “module” refer to software or a hardware component such as a field-programmable gate array (FPGA) or an application-specific integrated circuit (ASIC), and the unit or the module performs certain functions. However, the “unit” or “module” is not limited to software or hardware. Each operation of each module may be performed through one piece of hardware or may be performed through different pieces of hardware. An operation of a plurality of modules may be performed through one piece of hardware, and an operation of one module may be performed through a plurality of pieces of hardware.

According to an embodiment of the present invention, user-customized information can be generated by extracting at least a piece of visual information that the user wants to receive from various visual information such as shape information, spatial information, and complex information generated from an image.

According to an embodiment of the present invention, an improved sense of immersion can be provided to the user by converting extracted visual information into other sensory signals suitable for the characteristics of each piece of visual information. According to an embodiment of the present invention, an improved sense of immersion can be provided to the user by correcting the sensory signals converted from the extracted visual information according to the user's action.

According to an embodiment of the present invention, by synchronizing and transmitting a plurality of sensory signals converted from image information, it is possible to induce the user to more accurately perceive necessary visual information through a substitution sense.

In addition, since various pieces of visual information can be accurately transmitted through the present invention, the present invention can be applied not only to supporting for walking/living of the visually impaired, which was the scope of application of the conventional sensory substitution technology, but also to military operations requiring the transmission of confidential information and extreme work environments underground or late at night.

Further, according to the present invention, in a realistic virtual environment, augmented reality, or non-face-to-face society, the present invention can be applied to various services that allow users to experience visual information by transmitting the visual information more efficiently and realistically in a personalized way.

Effects of the present invention are not limited to the above-described effects and other effects that are not described may be clearly understood by those skilled in the art from the above detailed descriptions.

While the example embodiments of the present invention and their advantages have been described above in detail, it should be understood that various changes, substitutions and alterations may be made herein without departing from the scope of the invention as defined by the following claims.

### REFERENCE NUMERALS

100, 100′: APPARATUS FOR SENSORY SUBSTITUTION AND MULTI-PATH TRANSMISSION OF VISUAL INFORMATION
110: INPUT MODULE
111: USER INPUT UNIT
112: USER INPUT INFORMATION PROCESSING UNIT
113: IMAGE INPUT UNIT
120: SENSORY PATHWAY DETERMINATION MODULE
130: IMAGE INFORMATION DIVISION MODULE
131: SHAPE INFORMATION GENERATION UNIT
132: SPATIAL INFORMATION GENERATION UNIT
133: GLOBAL INFORMATION GENERATION UNIT
140, 140′: TARGET INFORMATION EXTRACTION MODULE
141: COMPLEX INFORMATION GENERATION UNIT
142: VISUAL INFORMATION EXTRACTION UNIT
150: SUBSTITUTION SIGNAL GENERATION MODULE
151: FIRST INFORMATION CONVERSION UNIT
152: SECOND INFORMATION CONVERSION UNIT
153: THIRD INFORMATION CONVERSION UNIT
160: CONTROL AND OUTPUT MODULE
161: TIME ANALYSIS UNIT
162: TRANSMISSION SIGNAL SYNCHRONIZATION UNIT
163: SUBSTITUTION SIGNAL CORRECTION UNIT
164: TRANSMISSION PATH SETTING UNIT
165: MULTI-PATH OUTPUT UNIT

What is claimed is:

1. An apparatus for sensory substitution and multi-path transmission of visual information, the apparatus comprising:
a target information extraction module configured to extract information of which a type corresponds to a type requested by a user from image information received from an external device on the basis of user input information;
a sensory pathway determination module configured to determine one or more sensory pathways through which the information extracted based on the user input information is transmitted;
a substitution signal generation module configured to convert the extracted information to generate substitution signals respectively corresponding to the one or more sensory pathways; and
a control and output module configured to output the substitution signals through a sensory signal transmission device of the user,
wherein the type is included in the user input information.

2. The apparatus of claim 1, wherein the one or more sensory pathways include a plurality of sensory pathways, and
the substitution signal generation module converts the extracted information to generate substitution signals respectively corresponding to the plurality of sensory pathways.

3. The apparatus of claim 2, wherein the plurality of sensory pathways include sensory pathways corresponding to at least two senses of a visual sense, an auditory sense, a tactile sense, and a heat sensation.

4. The apparatus of claim 1, wherein the target information extraction module analyzes the image information to generate overall visual information including at least one of shape information, spatial information, global information, and complex information, and extracts information of which a type corresponds to the type from the overall visual information on the basis of the user input information.

5. The apparatus of claim 2, wherein the control and output module synchronizes the substitution signals respectively corresponding to the plurality of sensory pathways on the basis of a frame of the image information, and outputs the synchronized substitution signals through the sensory signal transmission device.

6. The apparatus of claim 1, wherein the control and output module recognizes the user's action on the basis of the image information, corrects characteristics of the substitution signals on the basis of the user's action, and outputs the corrected substitution signals through the sensory signal transmission device.

7. The apparatus of claim 1, wherein the control and output module corrects characteristics of the substitution signals on the basis of a position of an input device of the image information and a position of the sensory signal transmission device, and outputs the corrected substitution signals through the sensory signal transmission device.

8. The apparatus of claim 1, wherein the user input information includes information on a transmission strength, and
the control and output module corrects a strength of the substitution signal according to the transmission strength, and outputs the corrected substitution signals through the sensory signal transmission device.

9. The apparatus of claim 1, wherein the control and output module calculates a time required for the substitution signal generation module to convert the extracted information into the substitution signals, and
the substitution signal generation module determines whether the conversion of the extracted information into the substitution signals is to be simplified on the basis of the time.

10. A method for sensory substitution and multi-path transmission of visual information, the method comprising:
extracting information of which a type corresponds to a type requested by a user from image information received from an external device on the basis of user input information;
determining one or more sensory pathways through which the information extracted based on the user input information is transmitted;
converting the extracted information and generating substitution signals respectively corresponding to the one or more sensory pathways; and
outputting the substitution signals through a sensory signal transmission device of the user.

11. The method of claim 10, wherein the one or more sensory pathways include a plurality of sensory pathways, and
in the generating of the substitution signals, the extracted information is converted and substitution signals respectively corresponding to the plurality of sensory pathways are generated.

12. The method of claim 11, wherein the plurality of sensory pathways include sensory pathways corresponding to at least two senses of a visual sense, an auditory sense, a tactile sense, and a heat sensation.

13. The method of claim 10, further comprising, after the generating of the substitution signal, a substitution signal synchronization operation of synchronizing the substitution signals on the basis of a frame of the image information.

14. The method of claim 10, further comprising, after the generating of the substitution signal, a substitution signal correction operation of correcting characteristics of the substitution signals on the basis of a position of an input device of the image information and a position of the sensory signal transmission device,
wherein, in the outputting of the substitution signal, the corrected substitution signals are output through the sensory signal transmission device.

15. The method of claim 10, wherein, in the extracting of the information of which the type corresponds to the type requested by the user,
the image information is analyzed and overall visual information including at least one of shape information, spatial information, global information, and complex information is generated, and
the information of which the type corresponds to the type is extracted from the overall visual information on the basis of the user input information.

16. The method of claim 11, wherein, in the outputting of the substitution signal, the substitution signals respectively corresponding to the plurality of sensory pathways are synchronized on the basis of a frame of the image information, and the synchronized substitution signals are output through the sensory signal transmission device.

17. The method of claim 10, wherein the user input information includes information on a transmission strength, and
in the generating of the substitution signal, a strength of the substitution signal is corrected by reflecting the transmission strength.

18. The method of claim 10, wherein, in the generating of the substitution signal,
a time required to convert the extracted information into the substitution signals is calculated, and
whether the conversion of the extracted information into the substitution signals is to be simplified is determined based on the time, the extracted information is converted based on a result of the determination, and the substitution signals are generated.
